(19) United States
(12) Patent Application Publication
(10) Pub. No.: US 2016/0220221 A1
(43) Pub. Date: Aug. 4, 2016

(54) Apparatuses And Methods For Determining The Beam Width Of A Computed Tomography Scanner

(71) Applicant: THE UAB RESEARCH FOUNDATION, BIRMINGHAM, AL (US)

(72) Inventors: David M. Gauntt, HOMEWOOD, AL (US); Gary T. Barnes, Vestavia Hills, AL (US)

(21) Appl. No.: 15/015,018

(22) Filed: Feb. 3, 2016

Related U.S. Application Data
(60) Provisional application No. 62/111,191, filed on Feb. 3, 2015.

(51) Int. Cl. A61B 6/00; A61B 6/03; A61B 6/06

(52) U.S. Cl. CPC A61B 6/58; A61B 6/06; A61B 6/032

(57) ABSTRACT

A method for determining a beam width of a computed tomography scanner includes calculating a dose length product DLP and dividing it by an accumulated radiation dose D(0). In some embodiments, DLP is calculated from measurements obtained using a pencil ionization chamber and a radiopaque mask that is slid over the chamber. In other embodiments, DLP is calculated from measurements obtained using a radiation dosimeter that is moved long a longitudinal axis of the scanner at a known velocity v.

### CROSS-REFERENCE TO RELATED APPLICATION

This application claims priority to co-pending U.S. Provisional Application Ser. No. 62/111,191, filed Feb. 3, 2015, which is hereby incorporated by reference herein in its entirety.

### BACKGROUND

Computed tomography (CT) is an imaging technology used in diagnostic radiology that uses x-rays to produce images that correspond to two-dimensional “slices” through a patient. FIG. 1 schematically illustrates a typical CT scanner 10. As shown in this figure, the scanner 10 includes a table 12 upon which the patient lies that can be passed through a circular gantry 14. An x-ray tube and an imaging radiation detector (not shown) are mounted on the gantry 14 and rotate around the patient as the table 12 passes through the gantry.

Such CT scanners normally use a “fan-beam” geometry in which the x-rays are restricted to a volume that is relatively wide in a transverse (left/right) direction but relatively narrow in longitudinal (head/foot) direction. FIG. 1 illustrates an example of such a beam 16 of radiation. The intensity of the radiation as a function D(x) of position x along the longitudinal axis of the scanner is referred to as the radiation profile. FIG. 2 is a graph that shows an example of a measured radiation profile. The width of the radiation profile is referred to as the “radiation profile width” or simply the “beam width.” This dimension is identified in FIG. 1. A typical CT scanner may have anywhere from a half dozen to a dozen different beam widths that can be used.

Measurement of the beam width is considered an important part of quality assurance programs for CT scanners. This is because only the central part of the beam is used to produce the CT images, while the full width of the beam contributes to the radiation dose of the patient. Thus, a beam that is wider than necessary imparts more radiation to the patient than is necessary. The American College of Radiology (ACR) has for several years recommended that the beam width of CT scanners be measured as part of an annual quality assurance program for all of the beam widths in clinical use.

The standard technique for measuring the beam width of a CT scanner is to expose film to the beam. Standard radiographic film may be used, although self-developing radiochromic film is more commonly used. The film is placed at the isocenter of the scanner (i.e., the intersection of the central plane of the radiation field and the axis of rotation of the gantry) and a CT exposure is made without moving the patient table. The x-ray radiation darkens a stripe on the film and the width of this stripe is equal to the beam width. The width of the stripe can be measured using a ruler or regular markings on the film, or by using a magnifier device with a graticule. However, because the edge of the stripe is invariably blurred, these measurements are subjective in nature and therefore imprecise. Although the width can be measured more precisely by using an optical scanner to digitize the image of the film and using image processing software to measure the width of the stripe, this procedure is much more difficult than the former methods.

One variation that has been proposed is to use computed radiographic (CR) plates in place of the film. Unlike film, the plates are reusable. This technique, therefore, does not consume any supplies. However, the technique involves greater effort than measuring a stripe on radiochromic film and cannot be used over as wide a range of radiation levels as film. The CR plates will saturate at all but the lowest radiation levels available on a typical CT scanner.

At least one manufacturer has developed an electronic device to accurately measure CT beam width. The device comprises a thin radiation detector that is placed on the patient table. The table is moved continuously during a CT scan and the radiation dose rate as a function of time is recorded on a computer. The dose rate can be converted to a measurement of the radiation dose profile as a function of position, and the beam width can be calculated from the dose profile. Although this provides an accurate measurement of the beam width, it is a more cumbersome procedure than using radiochromic film. In addition, the device is much more expensive than the other alternatives.

The pencil ionization chamber is one of the standard tools used by medical physicists when performing quality assurance tests on CT scanners. This device measures the total radiation integrated over a standardized length (e.g., 10 cm) along the longitudinal axis of the CT scanner gantry. In some embodiments, the pencil ionization chamber is a 0.5 inch diameter rod made of radiolucent plastic having a hollow, air-filled chamber inside. The pencil ionization chamber is connected to a radiation dosimeter, which measures the average air kerma (i.e., the radiation dose) in the chamber. The average air kerma is then multiplied by the length of the chamber, either automatically by the dosimeter or manually by the user, to determine the “dose-length product,” or DLP. The DLP is the integral of the radiation profile D(x) over the length of the ionization chamber:

DLP=∫D(x)dx

One technique that has been informally discussed is to use an established technique such as film to measure the beam width for a single collimation (beam width) setting, and then measure the DLP of the beam using a pencil ionization chamber for all available collimation settings. The ratio of the DLP for the first setting to the width of the beam measured using film is then used as a calibration constant that can be used to determine the beam width for other settings. This represents a significant simplification compared to using film to measure all beam widths, but still has the drawbacks associated with a single beam width measurement using film.

As can be appreciated from the above discussion, there is a need for an inexpensive and simple way to quickly and accurately determine the beam width of a CT scanner.

### BRIEF DESCRIPTION OF THE DRAWINGS

The present disclosure may be better understood with reference to the following figures. Matching reference numerals designate corresponding parts throughout the figures, which are not necessarily drawn to scale.

FIG. 1 is a schematic diagram of a typical computed tomography (CT) scanner.

FIG. 2 is a plot of an example radiation profile of a CT scanner.

FIG. 3A is an end view of a first embodiment of a mask that can be used in a method for determining the beam width of a CT scanner.

FIG. 3B is a side view of the mask of FIG. 3A.

FIG. 4A is a schematic view illustrating a pencil ionization chamber positioned at the isocenter of a CT scanner.

FIG. 4B is a schematic view illustrating the pencil ionization chamber positioned at the isocenter of a CT scanner when the mask of FIG. 3 is placed over the chamber.

FIGS. 5A and 5B are graphs that plot the radiation profile of a CT scanner measured by a pencil ionization chamber without and with the mask of FIG. 3, respectively.

FIG. 5C is a graph that plots the difference between the two graphs of FIGS. 5A and 5B.

FIG. 6A is an end view of a second embodiment of a mask that can be used in a method for determining the radiation profile width of a CT scanner.

FIG. 6B is a partial, cross-sectional side view of the mask of FIG. 6A.

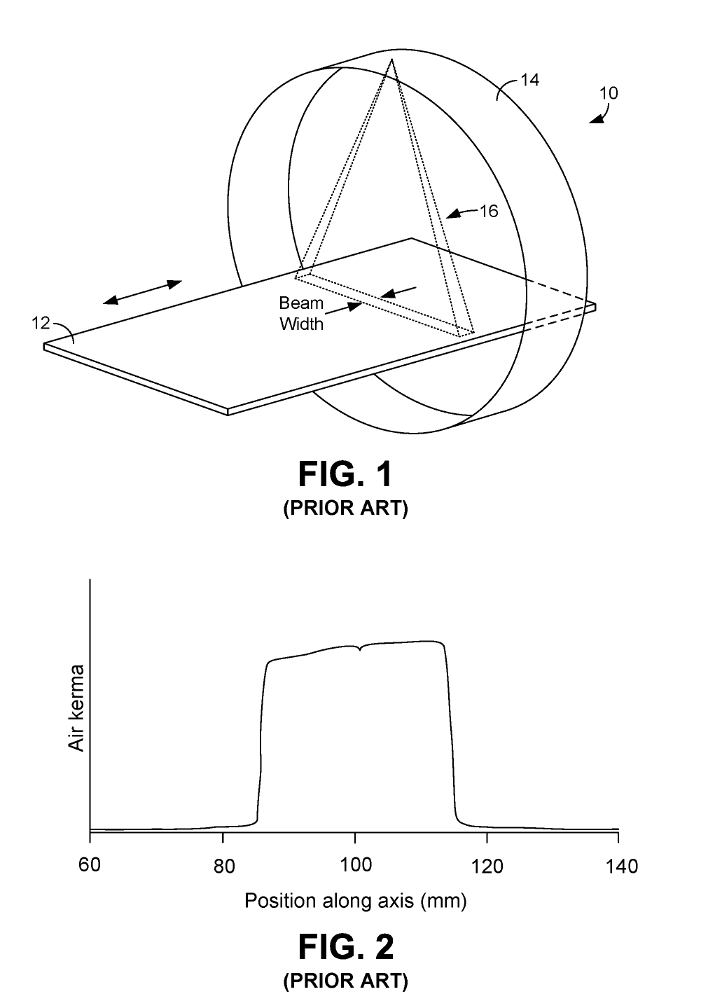

### DETAILED DESCRIPTION

As described above, there is a need for an inexpensive and simple way to quickly and accurately determine the beam width of a computed tomography (CT) scanner. Disclosed herein are apparatuses and methods for measuring beam width. The apparatuses include a radiopaque mask that can be placed over a pencil ionization chamber. In some embodiments, the radiation dose at the center of the x-ray beam is measured using the pencil ionization chamber both with and without the mask present. The beam width can then be calculated based upon these measurements and a further measurement performed at a different beam width setting.

In the following disclosure, various specific embodiments are described. It is to be understood that those embodiments are example implementations of the disclosed inventions and that alternative embodiments are possible. All such embodiments are intended to fall within the scope of this disclosure.

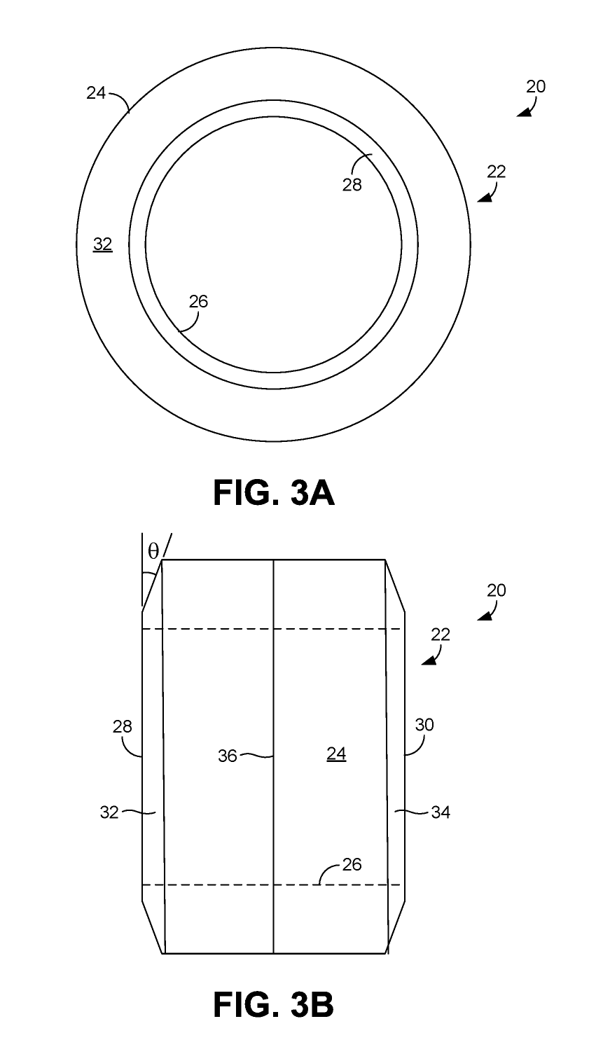

FIGS. 3A and 3B illustrate a first radiopaque mask 20 that can be used in conjunction with a pencil ionization chamber for the purpose of determining the beam width. More particularly, the mask 20 can be slid over the pencil ionization chamber when the radiation dose of the x-ray beam is measured using the pencil ionization chamber. As used herein, the term “radiation dose,” or simply “dose,” is used to refer to any radiation quantity that is proportional to the intensity of radiation of a given beam quality, whether exposure, air kerma, or absorbed dose. As is apparent from FIGS. 3A and 3B, the mask 20 comprises a continuous cylindrical body 22 that includes a cylindrical outer surface 24, a concentric cylindrical inner surface 26 (which defines a passage through which the pencil ionization chamber can pass), a first end surface 28, and a second end surface 30. In some embodiments, the end surfaces 28,30 are generally perpendicular to the inner surface 26 and the longitudinal direction of the body 22. The dimensions of the body 22 can depend upon the dimensions of the pencil ionization chamber with which the mask 20 is used. In some embodiments, the body 22 has an outer diameter (surface 24) of approximately 0.75 inches, an inner diameter (surface 26) of approximately 0.5 inches, and a length of approximately 0.4 inches (see FIG. 3B). Irrespective of its dimensions, the body 22 is made of a radiopaque material, such as tungsten. In some embodiments, the body 22 is hollow and is constructed of tungsten walls that are approximately 3 mm thick. The transmission of x-rays with photon energies typical of CT scanners through 3 mm of tungsten is much less than 0.1%.

With particular reference to FIG. 3B, the ends of the body 22 can comprise chamfers 32 and 34 that minimize the effects of a tilt error when positioning the mask 20 and pencil ionization chamber within the CT scanner. In some embodiments, the chamfers 32, 34 form an angle, θ (FIG. 3B), of approximately 6 degrees relative to the end surfaces 28, 30. As is further shown in FIG. 3B, the body 22 can include a visible centerline 36 that denotes the center of the mask 20 along its longitudinal direction and aids in the centering of the mask within the x-ray beam. Most CT scanners are equipped with alignment lasers that mark the central plane of the x-ray beam. When the line 36 is illuminated by the alignment laser, the mask 20 is centered in the beam.

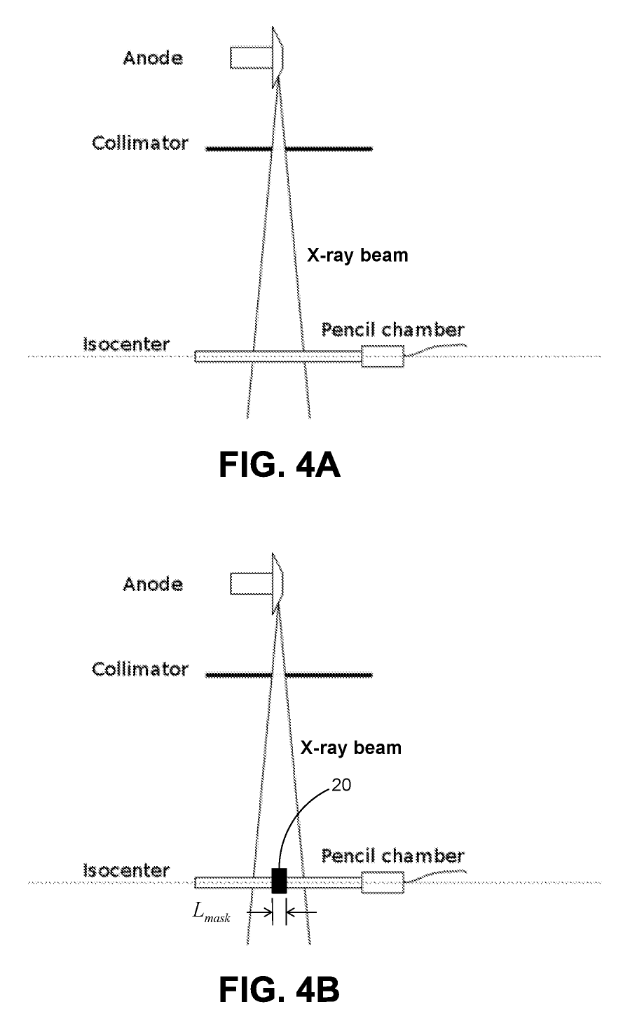

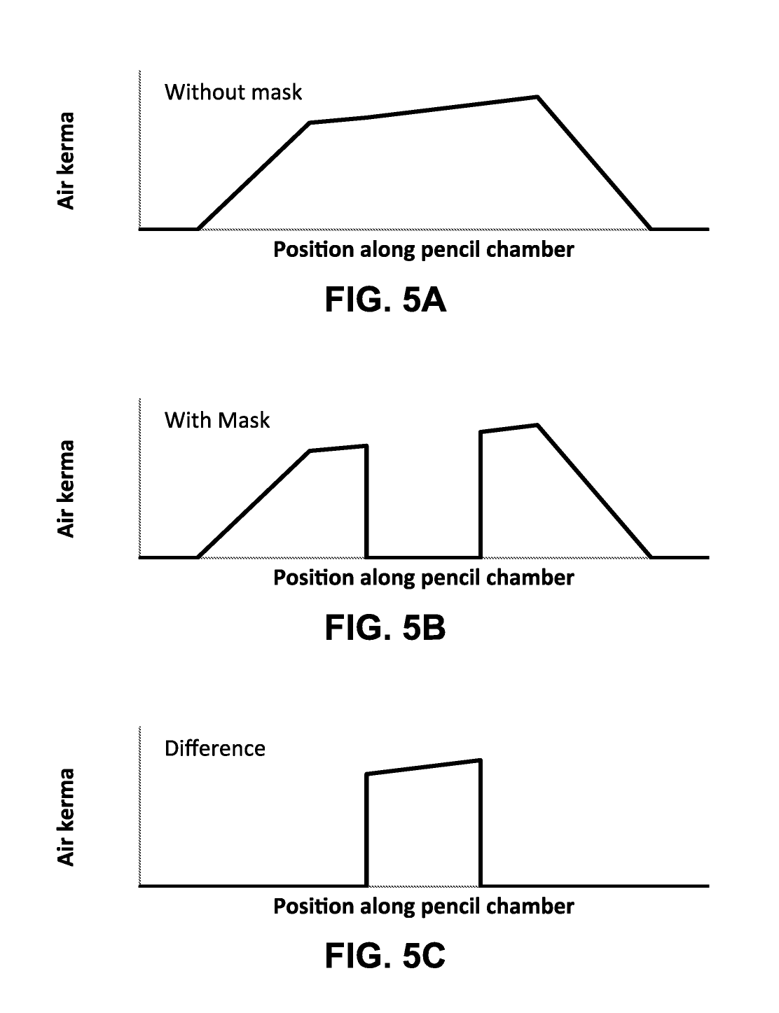

As noted above, the mask 20 can be used to determine the beam width of a CT scanner. To do this, the pencil ionization chamber can be placed at the isocenter of the gantry of the CT scanner such that it extends along the longitudinal axis of the gantry and the width direction of the x-ray beam when it is emitted. This is illustrated in FIG. 4A. The radiation dose within the pencil ionization chamber can then be measured during a brief x-ray exposure. FIG. 5A shows an example radiation profile during such a measurement. The DLP when the mask is not present, i.e., DLPₙₒₘₐₛₖ, is the integral over this profile.

Next, the mask 20 is slid onto the pencil ionization chamber and positioned at the center of the x-ray beam, as illustrated in FIG. 4B. At this point, the radiation dose within the pencil ionization chamber is measured during an x-ray exposure identical to the previous exposure. FIG. 5B shows an example radiation profile during such a measurement. The DLP when the mask if present, i.e., DLPₘₐₛₖ, is the integral over this profile.

Once these two measurements have been made, the accumulated radiation dose D(0) at the center of the radiation profile D(x) can be calculated by subtracting DLPₘₐₛₖ from DLPₙₒₘₐₛₖ and dividing the mask length, Lₘₐₛₖ:

$$D ( 0 ) = \frac{( {DLP}_{nomask} - {DLP}_{mask} )}{{L}_{mask}} \qquad ( Equation 1 )$$

FIG. 5C shows the difference between the two radiation profiles of FIGS. 5A and 5B. The value (DLPₙₒₘₐₛₖ−DLPₘₐₛₖ) is equal to the integral of this differential profile. The value at the center of the differential profile is equal to the integral divided by the length of the profile.

Once the value of D(0) has been determined, it is possible to determine the beam width W_{beam} for any nominal collimation (nominal beam width) by changing the collimation setting to a new setting (and therefore a new beam width) and acquiring a new DLP_{new} measurement without the mask. The beam width W_{beam} can then calculated by dividing the DLP_{new} by D(0)

$${W}_{beam} = \frac{{DLP}_{new}}{D ( 0 )} \qquad ( Equation 2 )$$

The above-described procedure requires less work than determining the beam width using any of the currently practiced methods. Experiments performed by the inventors indicate that the measured beam width is typically accurate to within 0.3 mm for a 40 mm wide beam, implying a 0.8% accuracy for the value of D(0).

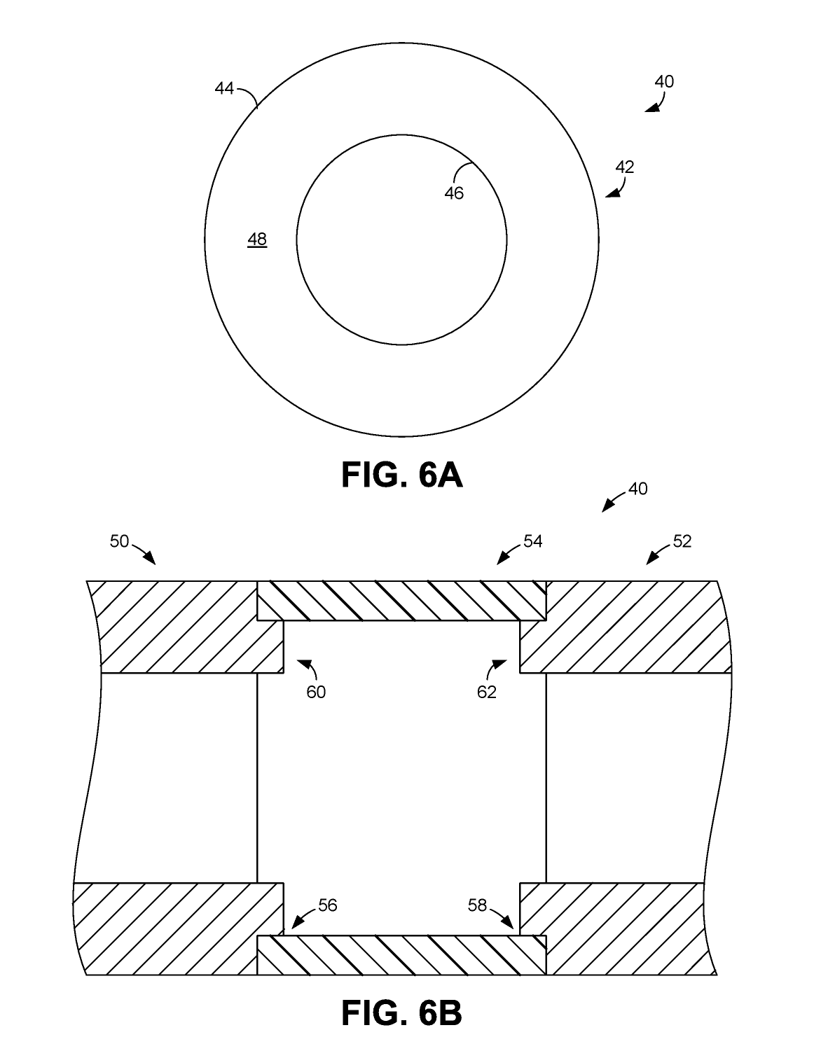

FIGS. 6A and 6B illustrate a second radiopaque mask 40 that can be used in conjunction with a pencil ionization chamber for the purpose of determining beam width. With reference to FIG. 6A, the mask 40 comprises a body 42 having a cylindrical outer surface 44, a concentric cylindrical inner surface 46 (which defines a passage through which the pencil ionization chamber can pass), a first end surface 48, and a second end surface (not visible). The dimensions of the body 42 can depend upon the dimensions of the pencil ionization chamber with which the mask 40 is used. In some embodiments, however, the body 42 has an outer diameter (surface 44) of approximately 0.75 inches, an inner diameter (surface 46) of approximately 0.375 inches, and a length of approximately 4 inches.

With reference to FIG. 6B, the body 42 can be formed by two radiopaque cylinders 50 and 52 that are joined by a central radiolucent cylindrical spacer 54. In some embodiments, the cylinders 50, 52 are made of tungsten and the spacer 48 is made of a polymer material, such as poly(methyl methacrylate) (PMMA). As shown in FIG. 6B, the cylinders 50, 52 can each have a circular outer notch 56, 58 that is adapted to receive an end of the spacer 54.

To measure the beam width using the mask 40, the mask is slid onto the pencil ionization chamber, positioned at the center of the x-ray beam, and the radiation dose within in the pencil ionization chamber is measured during an x-ray exposure. Because the only portion of the pencil ionization chamber that is exposed to the x-rays is the central part that aligns with the spacer 54, the accumulated radiation dose D(0) at the center of the radiation profile D(x) can be calculated by dividing the DLP with the mask 40 in place, i.e., DLPₘₐₛₖ, by Lₘₐₛₖ:

$$D ( 0 ) = \frac{{DLP}_{mask}}{{L}_{mask}} \qquad ( Equation 3 )$$

where Lₘₐₛₖ is the distance between the proximal ends 60, 62 of the cylinders 50, 52. Once the value of D(0) has been calculated, it is possible to determine the beam width W_{beam} for any nominal collimation (nominal beam width) by changing the collimation setting, acquiring a new DLP measurement, DLP_{new} without the mask, and dividing DLP_{new} by D(0) as in Equation 2. This procedure also requires less work than measuring the beam width using any of the currently practiced methods.

It is noted that an alternative approach would be to use a removable spacer. In such a case, a user could place the two radiopaque cylinders on the pencil ionization chamber and use the removable spacer to set the distance between them. The spacer could then be removed before taking the radiation dose measurement.

The approaches described above require a pencil chamber whose active length is substantially longer than the radiation profile width. The active length of the chamber is the length that is sensitive to radiation. The standard pencil chamber is 10 cm long, so the approaches described above may not suitable for radiation profiles substantially wider than 8 cm. However, the method may be adapted for wider beams by acquiring a series of measurements by longitudinally moving the pencil chamber a fixed distance from one measurement to another. As long as the total distance moved plus the length of the chamber is substantially wider than the radiation beam, the DLP of the beam can be determined from the measurements.

For example, assume that the radiation beam width is set to 16 cm and the active length of the pencil chamber is 10 cm. The DLP can be determined by taking radiation dose measurements for three different x-ray exposures. All three measurements can be taken with the longitudinal axis of the pencil chamber coincident with the longitudinal axis of the scanner. The first exposure can be taken with the center of the pencil chamber 10 cm from the isocenter, the second exposure can be taken at the isocenter, and the third exposure can be taken 10 cm from the isocenter on the opposite side of the scanner from the first exposure. In such a case, the DLP is the sum of these three measurements. Alternately, five measurements can be taken by moving the chamber 5 cm from one measurement to the next and then dividing the sum of the five dose measurements by 2.

Another approach that would work with such wide profiles would be to measure D(0) directly by using a radiation dosimeter whose active length is significantly less than the width of the radiation profile, and then measure the DLP of the full radiation profile. The DLP could be measured by using the same dosimeter used to measure D(0), but move the dosimeter along the gantry axis at a fixed known velocity v. If the dose rate at position x is equal to \dot{D}(x) during an exposure of duration T, then the accumulated dose at any position x is equal to

D(x)=T\dot{D}(x) (Equation 4)

and the accumulated dose at isocenter is

D(0)=T\dot{D}(0) (Equation 5)

The exposure accumulated by the dosimeter moving through the radiation profile at a constant velocity v is:

$$\begin{matrix}{D}_{v} = {\int }_{- T / 2}^{+ T / 2} \dot{D} ( vt ) d t & ( Equation 6 ) \\ = {\int }_{- T / 2}^{+ T / 2} \dot{D} ( x ) d \frac{x}{v} & ( Equation 7 ) \\ = \frac{1}{v} {\int }_{- vT / 2}^{+ vT / 2} \dot{D} ( x ) d x & ( Equation 8 ) \\ = \frac{1}{vT} {\int }_{- vT / 2}^{+ vT / 2} D ( x ) d x & ( Equation 9 )\end{matrix}$$

If the exposure is long enough that the product vT is substantially larger than the radiation profile width, then

$$\begin{matrix}{D}_{v} = \frac{1}{vT} {\int }_{- ∞}^{+ ∞} D ( x ) d x & ( Equation 10 ) \\ {D}_{v} = \frac{DLP}{vT} & ( Equation 11 )\end{matrix}$$

and the radiation profile width can be calculated using the following equation:

$$W = \frac{DLP}{D ( 0 )} = \frac{{D}_{v} vT}{D ( 0 )} \qquad ( Equation 12 )$$

This derivation assumes that the active length of the radiation dosimeter is infinitesimally small; however, the result is the same for any radiation dosimeter with an active length substantially smaller than the beam width. The radiation dosimeter may be either an ionization chamber or a solid state sensor or any sensor capable of measuring a radiation exposure or dose.

This technique differs from the use of the electronic device to accurately measure CT beam width that exists in the prior art in that there is no need to measure and store the dose rate as a function of time to determine a dose profile. All that the dosimeter needs to do is measure the accumulated dose over the length of an exposure.

1. A method for determining a beam width of a computed tomography scanner, the method comprising:
positioning a pencil ionization chamber at an isocenter of the scanner and exposing the pencil ionization chamber to radiation emitted by the scanner;
measuring the radiation within the pencil ionization chamber and calculating a dose-length product DLP based upon the measured radiation dose;
calculating an accumulated radiation dose D(O) based upon the DLP;
changing a collimation setting of the scanner and again exposing the pencil ionization chamber to radiation emitted by the scanner;
measuring the radiation within the pencil ionization chamber and calculating a new dose-length product DLP_{new} based upon the measured radiation dose; and
calculating the beam width based by dividing DLP_{new} by D(0).

2. The method of claim 1, wherein positioning a pencil ionization chamber comprises positioning the pencil ionization chamber within the scanner with a radiopaque mask positioned over the pencil ionization chamber at the scanner isocenter and wherein calculating a dose-length product DLP comprises calculating the dose-length product with the mask DLPₘₐₛₖ.

3. The method of claim 2, wherein the mask comprises a single cylindrical body made of a radiopaque material.

4. The method of claim 3, further comprising positioning the pencil ionization chamber at the isocenter without the mask in place, exposing the pencil ionization chamber to radiation emitted by the scanner, measuring the radiation within the pencil ionization chamber, and calculating a dose-length product without the mask DLPₙₒₘₐₛₖ based upon the measured radiation dose.

5. The method of claim 4, wherein calculating an accumulated radiation dose D(0) comprises subtracting DLPₘₐₛₖ from DLPₙₒₘₐₛₖ and dividing the result by the length of the pencil ionization chamber.

6. The method of claim 2, wherein the radiopaque mask comprises two radiopaque cylinders that are spaced from each other by a radiolucent gap.

7. The method of claim 6, wherein calculating an accumulated radiation dose D(0) comprises dividing DLPₘₐₛₖ by the length of the radiolucent gap.

8. A radiopaque mask adapted to slide onto a pencil ionization chamber, the mask comprising:
a cylindrical body having an outer cylindrical surface, an inner cylindrical surface, a first end surface, and a second end surface, wherein the body is hollow and is made of a radiopaque material.

9. The mask of claim 8, wherein the body is made of tungsten.

10. The mask of claim 8, wherein ends of the body are chamfered.

11. The mask of claim 10, wherein the ends are chamfered at an angle of approximately 6 degrees.

12. A radiopaque mask adapted to slide onto a pencil ionization chamber, the mask comprising:
first and second radiopaque cylinders; and
a radiolucent spacer that joins the two radiopaque cylinders and creates a radiolucent gap of known length between the cylinders.

13. The mask of claim 12, wherein the radiopaque cylinders are made of tungsten.

14. The mask of claim 12, wherein the radiolucent spacer is cylindrical.

15. The mask of claim 12, wherein the radiolucent spacer is made of a polymer material.

16. The mask of claim 12, wherein the radiolucent spacer is made of poly(methyl methacrylate).

17. The mask of claim 12, wherein the radiolucent spacer is removable.

18. A method for determining a beam width of a computed tomography scanner, the method comprising:
emitting radiation from the scanner and measuring an accumulated radiation dose D(0) using a radiation dosimeter;
emitting radiation from the scanner a second time and measuring a radiation dose Dᵥ using the radiation dosimeter while moving the dosimeter along a longitudinal axis of the scanner at a known velocity v;
calculating a dose-length product DLP based upon the measured radiation dose Dᵥ; and
calculating the beam width by dividing DLP by D(0).

19. The method of claim 18, wherein calculating a dose-length product DLP comprises multiplying Dv by v and by a duration of the measurement T.

20. The method of claim 18, wherein the radiation dosimeter has an active length that is less than the beam width.
